(19) United States
(12) Plant Patent Application Publication
(10) Pub. No.: US 2023/0189676 P1
(43) Pub. Date: Jun. 15, 2023

(54) Apple tree named 'SF118'

(50) ;

(71) Applicant: The New Zealand Institute for Plant and Food Research Limited, Auckland (NZ)

(72) Inventors: Bruno ESSNER, Angers (FR); Kenneth Charles BREEN, Hastings (NZ)

(73) Assignee: The New Zealand Institute for Plant and Food Research Limited, Auckland (NZ)

(21) Appl. No.: 17/803,704

(22) Filed: Oct. 14, 2022

Related U.S. Application Data
(60) Provisional application No. 63/273,696, filed on Oct. 29, 2021.

(51) Int. Cl. A01H 6/74

(52) U.S. Cl. PLT/161

(57) ABSTRACT

A new and distinct variety of apple tree is described. The variety results from a limb sport found in Angers, France. The fruit of the apple tree of this new variety have an attractive appearance, characterised by a high proportion of red over color and an over color pattern that is a solid flush.

### CROSS REFERENCE TO RELATED APPLICATION

This application claims the benefit of U.S. Provisional Application No. 63/273,696, filed Oct. 29, 2021, the disclosure of which is incorporated by reference in its entirety.

Genus and species plant named: Malus domestica.

Variety denomination: ‘SF118’.

### BACKGROUND OF THE INVENTION

The new variety of apple tree ‘SF118’, Malus domestica, resulted from a spontaneous limb sport mutation of ‘Scifresh’ (U.S. Plant Pat. 13,888). The ‘Scifresh’ tree containing the limb sport of the new variety was discovered in 2012, growing in a cultivated area in Angers, France. It was selected for further observation because of its fruit skin over color. It was originally given the code SF118-29 but later named ‘SF118’. ‘SF118’ was first asexually propagated in 2012 in Angers, France by budding onto ‘M9’ rootstock. The grafted trees were planted in an orchard in 2014. The resulting plants were found to be true to type demonstrating that the characteristics of the new variety ‘SF118’ are stable and transmitted without change through succeeding generations.

### SUMMARY OF THE INVENTION

The new and distinct ‘SF118’ variety is characterised by its medium sized fruit and skin over color and pattern. The over color is red (near dark red 46B), the relative area of the over color large and the over color pattern a solid flush.

‘SF118’ is distinguished from its parent, ‘Scifresh’ by the following characteristics.

‘SF118’ has on average a higher percentage of the fruit skin color (83%) compared to ‘Scifresh’ 73%. In addition, ‘SF118's over color pattern is solid flush compared to ‘Scifresh’ which is solid flush with narrow, weakly defined stripes.

### BRIEF DESCRIPTION OF DRAWINGS

The accompanying photographs show typical specimens of the tree, foliage and fruit of the new variety as depicted in colors as nearly true as is reasonably possible to make the same in a color illustration of this character.

FIG. 1: ‘SF118’ fruit lateral views of a) highly colored fruit side and b) lower colored fruit side of the same fruit.

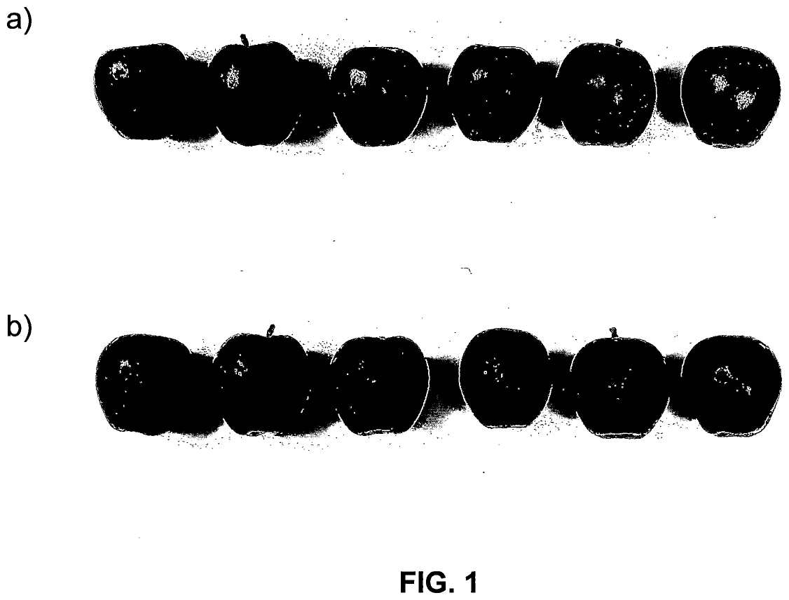

FIG. 2: ‘SF118’ longitudinal section of cut fruit.

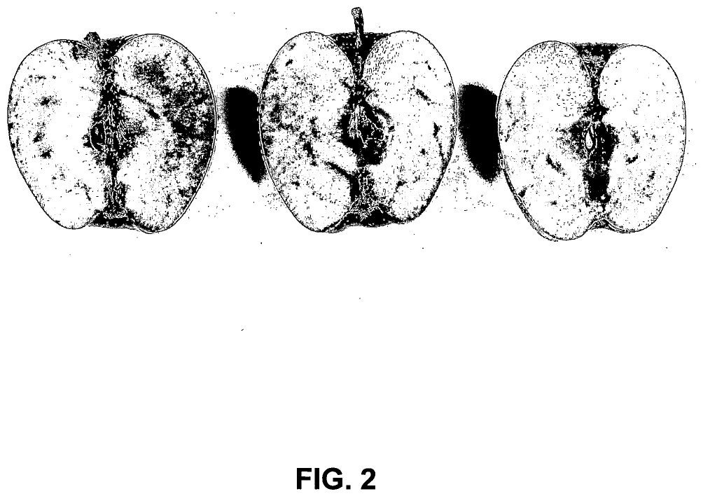

FIG. 3: ‘SF118’ 5-year-old tree.

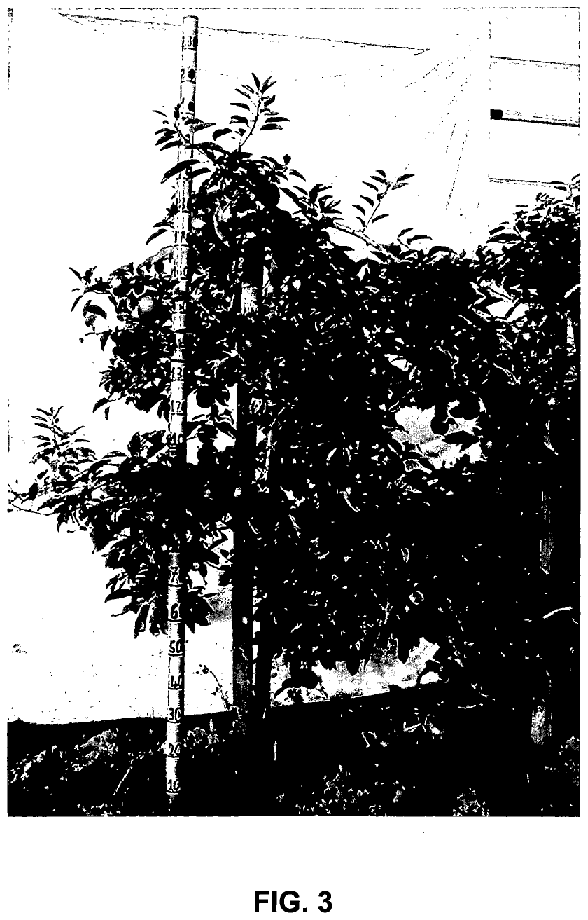

FIG. 4: ‘SF118’ top side of leaves.

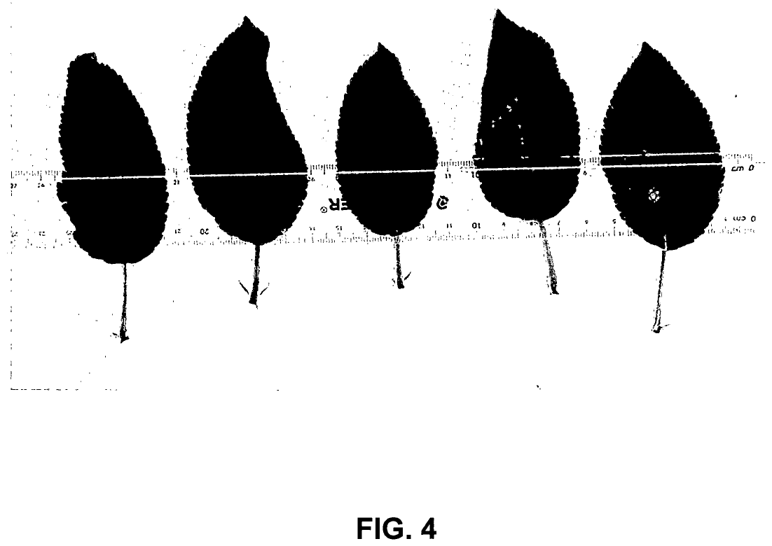

FIG. 5: ‘SF118’ flowers.

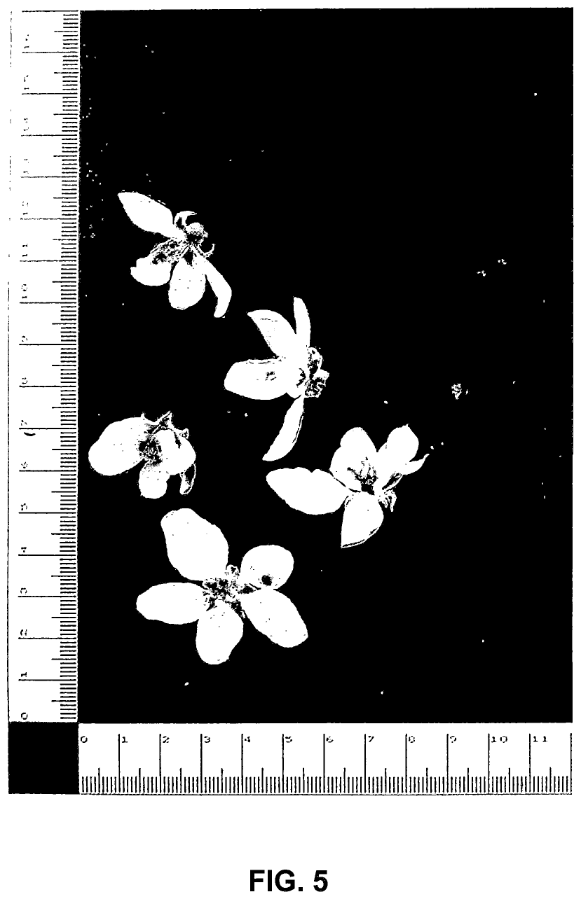

### DETAILED DESCRIPTION

The following is a description of the new variety with color terminology in accordance with The Royal Horticultural Society Color Charts (R.H.S.C.C.) 2015 edition. Observations made on trees in Angers, France; over two seasons, 2019 and 2020, aged 5-6 years on ‘M9’ rootstock; Measurements and observations taken from the five trees or plant parts taken off each of the five trees.
Tree:
   Size.—Average height 2.44 m, average width 1.68 m.
   Type.—Ramified.
   Habit.—Spreading.
   Vigour.—Medium to strong on ‘M9’, managed under standard orchard practices.
   Bearing.—On spurs and long shoots.
Trunk:
   Size.—Average circumference 20 cm above the graft union 15.9 cm.
   Bark texture.—Slightly rough.
   Bark color.—Near dark grey, 197C.
One-year-old shoot:
   Growth.—Straight.
   Thickness.—Medium, average 5-6 mm.
   Length of internode.—Medium, average 20 mm.
   Length of shoot.—Average 294 mm.
   Predominant color on sunny side.—Near medium brown, 200A.
   Pubescence on distal half of the shoot.—Medium.
   Number of lenticels.—Medium, average 7 per cm2.
   Lenticel shape.—Elliptical to irregular round.
   Lenticel size.—Average length 1 mm, average width 1 mm.
   Lenticel color.—Near light grey, 156C.
Vegetative bud:
   Shape of apex of vegetative bud.—Acute.
   Position of vegetative bud in relation to shoot.—Adpressed to slightly held out.
   Vegetative bud length.—Average 7 mm.
   Vegetative bud color.—Near dark brown, 200A.
Leaf blade:
   Attitude in relation to shoot.—Outwards.
   Length.—Medium, average 78 mm.
   Width.—Medium, average 40 mm.
   Ratio length/width.—Medium.
   Shape of base.—Obtuse.
   Shape of apex (excluding pointed tip).—Acute.
   Intensity of green color.—Medium.
   Incisions of margin (upper half).—Crenate.
   Lower surface pubescence.—Weak.
   Upper surface color.—Near dark brown green, 147A.
   Lower surface color.—Near medium brown green, 148C.
   Venation color.—Near medium grey green, 145C.
Petiole:
   Petiole length.—Medium, average 26 mm.
   Petiole width.—Average 3 mm.
   Petiole upper color.—Near light green, 139D.
   Petiole lower color.—Near medium grey green, 145C.
   Extent of anthocyanin coloration from base.—Medium.
   Presence of stipules.—Present.
   Stipule length.—Average 4 mm.
   Stipule width.—Average 1 mm.
   Stipule upper color.—Near medium brown green, 147B.
   Stipule lower color.—Near light green, 138C.
Flower:
   Predominant color at balloon stage.—Near red pink, 53C.
   Diameter with petals pressed into horizontal.—Medium, average 39 mm.
   Arrangement of petals.—Intermediate.
   Position of stigmas relative to anthers.—Above.
   Location of flower bud.—Mainly on spurs.
   Average Number of buds per spur.—1.2.
   Average Number of flowers per cluster.—5.
   Average number of sepals/flower.—5.
   Sepal length.—Medium, average 7 mm.
   Sepal width.—Average 4 mm.
   Sepal color.—Near light green, 145A.
   Attitude of sepals in relation to corolla.—Recurved.
   Average number of petals/flower.—5.
   Size of petal.—Average length 18 mm, average width 11 mm.
   Shape of petal (excluding the claw).—Ovate.
   Shape of base of petal (excluding the claw).—Rounded.
   Length of claw of petal.—Average 2 mm.
   Petal color on lower side.—Near medium purple red, 61C.
   Petal venation color on lower side.—Near medium purple 71B.
   Average number of stamen per flower.—19.
   Average filament length.—5 mm.
   Filament color.—Near white, 155D.
   Average anther length.—3 mm.
   Average anther width.—1 mm.
   Anther color.—Near light yellow, 4D.
   Pollen color.—Near medium yellow, 7D.
   Average pistil length.—9 mm.
   Pistil color.—Near light Green, 145B.
   Average pedicel length.—17 mm.
   Average pedicel width.—2 mm.
   Pedicel color.—Near dark green brown, 152B.
   Fragrance.—Present.
Young fruit:
   Extent of anthocyanin.—Small.
Fruit size:
   Size.—Medium, average weight 206 g.
   Height.—Medium, average 74 mm.
   Diameter.—Medium, average 74 mm.
   Position of maximum diameter.—Middle.
Fruit shape:
   General shape.—Conic to globose, most fruit tending towards being conic.
   Position of maximum diameter.—Middle.
   Ribbing.—Absent or weak.
   Crowning at calyx end.—Absent or weak.
   Size of eye.—Small to medium.
   Length of sepal.—Medium, average 4 mm.
   Depth of eye basin.—Medium, average 11 mm.
   Width of eye basin.—Medium, average 29 mm.
Fruit skin:
   Bloom of skin.—Absent or weak.
   Greasiness of skin.—Absent or weak.
   Ground color of skin.—Near medium yellow, 2A.
   Relative area of over color.—Large, average 83%.
   Hue of over color.—With bloom removed — Near dark red, 46B.
   Intensity of over color.—Medium.
   Pattern of over color.—Only solid flush.
   Area of russet around stalk attachment.—Absent.
   Area of russet on cheeks.—Absent.
   Area of russet around eye basin.—Absent.
   Size of lenticels.—Small.
   Surface texture of skin.—Smooth.
Fruit stalk:
   Length of stalk.—Medium, average 19 mm.
   Thickness of stalk.—Medium, average 4 mm.
   Depth of stalk cavity.—Medium, average 14 mm.
   Width of stalk cavity.—Medium, average 32 mm.
Fruit flesh:
   Firmness of flesh.—Firm, average 9.6 kgf.
   Color of flesh.—Yellowish, near 158A.
   Aperture of locules (in transverse section).—Closed or slightly open.
   Texture of flesh.—Medium.
   Water-core of flesh.—Absent to very weak.
Fruit flavour: Medium sweetness, average soluble solids, 13% brix, medium acidity.
Fruit seeds:
   Average number of locules.—5.
   Seed shape.—Ovate.
   Average seed length.—9 mm.
   Average seed width.—5 mm.
   Average seed weight.—0.06 g.
   Seed color.—Near medium brown, 175A.
Event timing:
   Time of beginning of flowering.—Medium, mid May, similar to ‘Scifresh’.
   Time for harvest.—Late, late September, similar to ‘Scifresh’.

TABLE 1
Comparison table between the similar varieties ‘Scifresh’ (parent),
‘SF1’ and the candidate variety ‘SF118’. Observations made on trees in
Angers, France; over 2 seasons, 2019 and 2020, on ‘M9’ rootstock.

| Characteristic | ‘Scifresh’ | ‘SF1’ | ‘SF118’ |
|---|---|---|---|
| Relative area | Medium, | Very Large, | Large, |
| of over color | average 73% | average 96% | average 83% |
| Pattern of | Solid flush with | Solid flush with | Only solid flush |
| over color | narrow, weakly | narrow, weakly | |
| | defined stripes | defined stripes | |
| Over color hue | Near 42D | Near 53A | Near 46B |
| with bloom | | | |
| removed | | | |
| Ground color | Yellow green, | Yellow, Near 4A | Yellow, Near 2C |
| of Skin | Near 154D | | |

1. A new and distinct variety of apple tree substantially as illustrated and described herein.
